SEQUENCE LISTING (9 sequences)

SEQ ID NO: 1 - DNA, 22 bp, artificial
  synthetic sequence
tagcagcacgtaaatattggcg

SEQ ID NO: 2 - DNA, 114 bp, artificial
  synthetic sequence
gtactagcagcacgtaaatattggcggctagctagcagcacgtaaatattggcggtcagctagcagcacgtaaatattggcgagctgctagcagcacgtaaatattggcgcgta

SEQ ID NO: 3 - DNA, 22 bp, artificial
  synthetic sequence
caaacaccattgtcacactcca

SEQ ID NO: 4 - DNA, 114 bp, artificial
  synthetic sequence
gtaccaaacaccattgtcacactccagctagctagcagcacgtaaatattggcggtcagccaaacaccattgtcacactccaagctgctagcagcacgtaaatattggcgcgta

SEQ ID NO: 5 - DNA, 212 bp, artificial
  synthetic sequence
gtaagtgccgtgtgtggttcccgcgggcctggcctctttacgggttatggccaaacaccattgtcacactccagctagctagcagcacgtaaatattggcggtcagccaaacaccattgtcacactccaagctgctagcagcacgtaaatattggcgccttgcgtgccttgaattactgacactgacatccactttttctttttctccacag

SEQ ID NO: 6 - DNA, 218 bp, artificial
  synthetic sequence
gtaagtgccgtgtgtggttcccgcgggcctggcctctttacgggttatggccaaacaccattgtcacactccagctagctagcagcacgtaaatattggcggtcagccaaacaccattgtcacactccaagctgctagcagcacgtaaatattggcgccttgcgtgccttgaattactgatttattcactgacatccactttttctttttctccacag

SEQ ID NO: 7 - DNA, 212 bp, artificial; identical to SEQ ID NO: 5
  synthetic sequence

SEQ ID NO: 8 - DNA, 54 bp, artificial
  synthetic sequence
gtactagcagcacgtaaatattggcggctagctagcagcacgtaaatattggcg

SEQ ID NO: 9 - DNA, 106 bp, artificial
  synthetic sequence
taattctaggcgactagtaaacaaacaaagacgtatgagactgactgaaacgccgtctcactgatgaggccatggcaggccgaaacgtcaaaaagaaaaataaaaa

(19) United States
(12) Patent Application Publication
(10) Pub. No.: US 2022/0106609 A1
(43) Pub. Date: Apr. 7, 2022

(54) PARAVOVIRAL VECTORS AND METHODS OF MAKING AND USE THEREOF

(71) Applicant: NIKEGEN, LLC, Dresher, PA (US)

(72) Inventors: Weidong XIAO, Dresher, PA (US); Xiangping YU, Fujian (CN)

(21) Appl. No.: 17/310,424

(22) PCT Filed: Dec. 16, 2020
(86) PCT No.: PCT/US2020/065329

§ 371 Date: Aug. 2, 2021

Related U.S. Application Data
(60) Provisional application No. 62/949,052, filed on Dec. 17, 2019.

(51) Int. Cl. C12N 15/86; C12N 15/11

(52) U.S. Cl. CPC C12N 15/86; C12N 15/111; C12N 2310/111; C12N 2750/14143; C12N 2330/51

(57) ABSTRACT

A recombination parvovirus vector that comprises a parvovirus capsid and a double-stranded vector genome having a sense-strand and an antisense-strand. The sense-strand comprises in the 5′ to 3′ direction: a parvovirus terminal repeat at the 5′ end a coding sequence of a gene of interest (GOI); and a parvovirus terminal repeat at the 3′ end. The vector genome further comprises a RNA destabilization/destruction domain (RDDD).

This application claims priority of U.S. Provisional Application No. 62/949,052, filed on Dec. 17, 2019.

### FIELD

The present invention generally relates to vectors for use in gene transfer and gene therapy applications. In particular, the present application relates to management of undesired antisense RNAs and double stranded RNAs (dsRNAs) produced during the production of recombinant viral vectors, such as parvovirus vectors.

### BACKGROUND

Recombinant parvoviruses, such as adeno associated viruses (AAV), have been widely used as gene transfer vectors in the field of gene therapy. However, recombinant parvoviruses often produce antisense RNAs or dsRNAs during replication, which results in reduced yield of viral production.

Therefore, there exists a need for new parvoviral vectors that can be produced with high yield.

### SUMMARY

The present application describes parvovirus vectors with a viral genome having an antisense RNA or dsRNA destabilization/destruction domain (RDDD), and includes methods and DNA constructs for producing such vectors. The constructs, vectors and methods are applicable to gene transfer/therapy applications, including those requiring delivery of recombinant gene expression cassettes.

One aspect of the present application relates to a parvovirus vector comprising: a parvovirus capsid; and a double-stranded vector genome comprising a sense-strand and an antisense-strand, wherein the sense-strand comprises in the 5′ to 3′ direction: a parvovirus terminal repeat at the 5′ end; a coding sequence of a gene of interest (GOI); and a parvovirus terminal repeat at the 3′ end, wherein the vector genome further comprises a RDDD.

In some embodiments the RDDD is located in the antisense-strand. In some embodiments, the RDDD is located in an intron of the GOI in a reverse orientation.

In some embodiments, the parvovirus vector is an AAV vector.

In some embodiments, the RDDD comprises a microRMA. In some embodiments, the RDDD comprises SEQ ID NO:1 and/or SEQ ID NO:3. In some embodiments, the RDDD comprises two or more copies of SEQ ID NO:1 and/or two or more copies of SEQ ID NO:3. In some embodiments, the RDDD comprises the nucleotide sequence selected from the group consisting of SEQ ID NOS: 2, 4, 5, 6 and 7.

In some embodiments, the RDDD comprises a ribozyme. In some embodiments, the ribozyme comprises SEQ ID NO:9.

In some embodiments, the parvovirus vector further comprises a second RDDD. In some embodiments, the second RDDD comprises SEQ ID NO:8

Another aspect of the present application relates to a method for improving production yield of a recombinant parvovirus, comprising the steps of: inserting a RNA destabilization/destruction domain (RDDD) into the recombinant parvovirus. The recombinant parvovirus comprises: a parvovirus capsid; and a double-stranded viral genome comprising a sense-strand and an antisense-strand, wherein the sense-strand comprises in the 5′ to 3′ direction: a parvovirus terminal repeat at the 5′ end; a coding sequence of a gene of interest (GOI); and a parvovirus terminal repeat at the 3′ end, wherein the RDDD is inserted into the viral genome.

In some embodiments, the RDDD is located in the antisense-strand of the viral genome. In some embodiments, the RDDD is located in an intron of the GOI in a reverse orientation.

### BRIEF DESCRIPTION OF FIGURES

FIG. 1 shows a cryptic promoter in the antisense orientation of a gene of interest (GOI) that drives the expression of antisense RNA from the antisense strand of the GOI expression cassette, which can negatively affect GOI mRNA expression and activity.

FIG. 2 shows dsRNA formation mechanisms in a parvovirus vector. Pathway A shows formation of GOI mRNAs from a recombinant parvovirus vector containing a complete genome encoding the full transcript unit for GOI, that may not have antisense transcription in the negative strand. Pathway B shows formation of dsRNAs from defective interfering (DI) particles containing a portion of the recombinant parvovirus vector genome. The resulting dsRNAs can then interfere with the expression and activity of the GOI mRNA.

FIG. 3 shows RNAi, ribozyme and antisense oligonucleotides that control or regulate the expression of mRNAs or translation therefrom. These tools are used in the parvovirus vectors of the present application to regulate antisense RNAs and dsRNAs formed from parvovirus vectors.

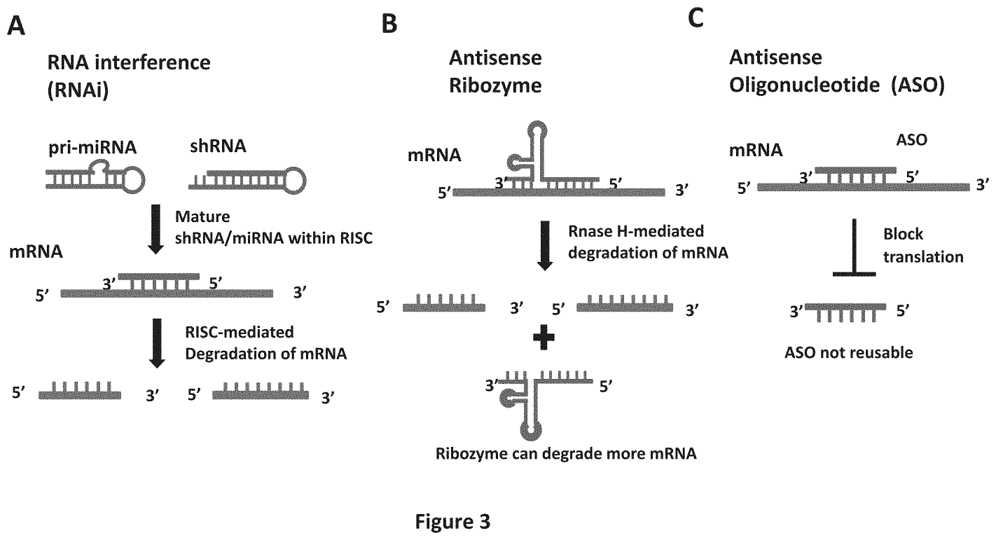

FIG. 4 shows embodiments of RNA destabilization/destruction domains (RDDDs) that reduce or eliminate the negative effects of antisense RNA. The RDDD is placed in the region before the transcription termination site or 3′ ITR in the antisense strand of the GOI with a cryptic promoter. The antisense RNA is destabilized or destroyed by RNA destabilization/destruction execution molecules (RDDDEMs) (right panel), while the GOI mRNA is not affected (left panel).

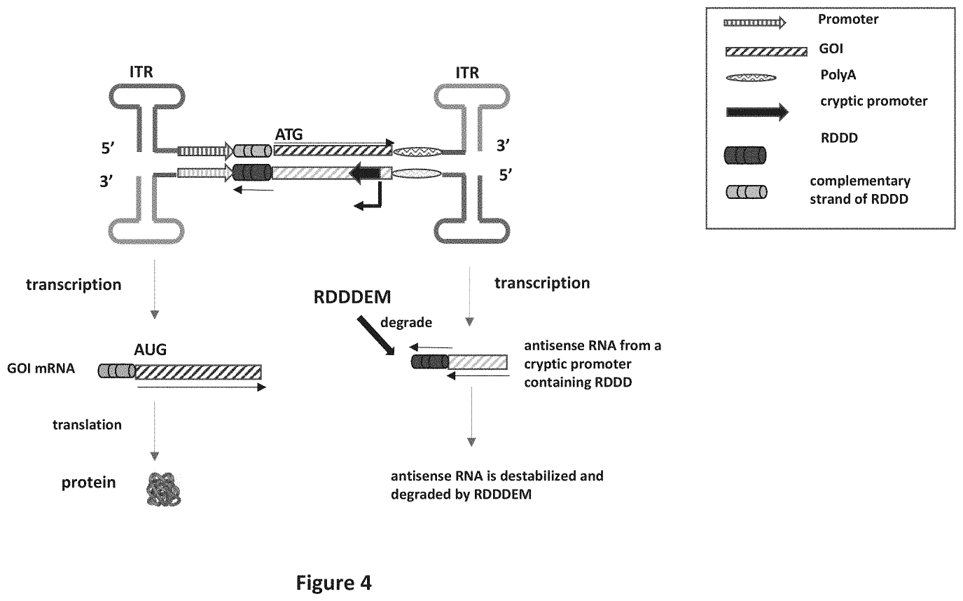

FIG. 5 depicts an exemplary parvoviral vector containing an RDDD to reduce or eliminate the effects of dsRNAs that may be formed from the parvoviral vector. The RDDD is placed before the transcription termination site or 3′ ITR of the antisense strand of the GOI. In this case, the dsRNAs expressed from the defective interference particles are destabilized or destroyed by RDDDEMs (right panel), while the GOI mRNA is not affected (left panel). The location of the RDDD can vary as long as it is only transcribed from DI particles, not the full length vectors.

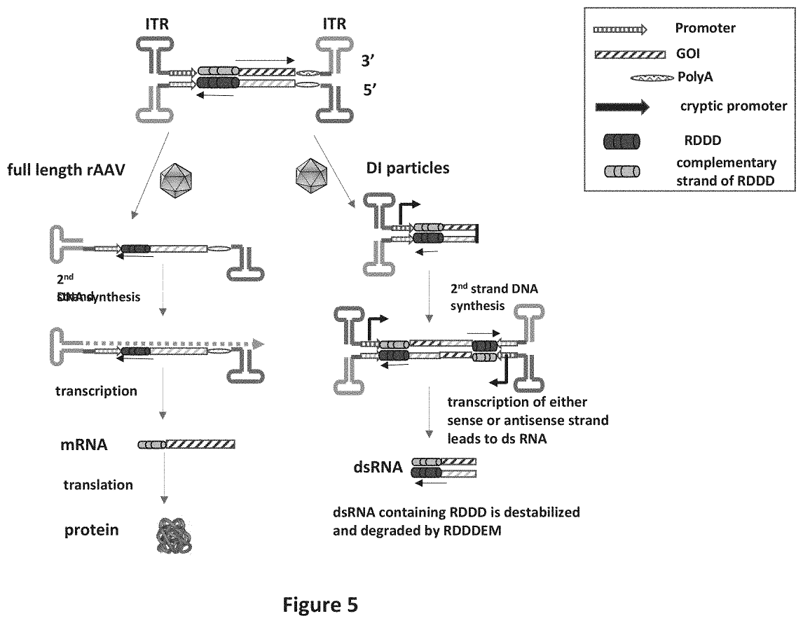

FIG. 6 illustrates an exemplary strategy for using a self-cleaving ribozyme to reduce or eliminate the effects of antisense RNAs. The ribozyme is placed in the region before the transcription termination site or 3′ ITR in the antisense strand of the GOI with a cryptic promoter. The antisense RNA is destabilized or destroyed by the ribozyme (right panel), while the GOI mRNA is not affected (left panel).

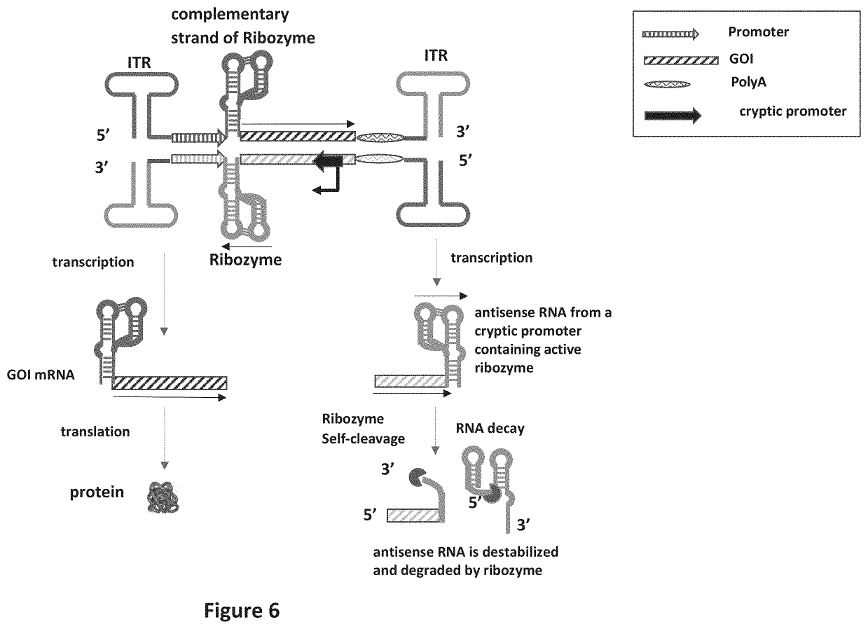

FIG. 7 illustrates an exemplary strategy for using a self-cleaving ribozyme to reduce or eliminate the effects of dsRNAs. The ribozyme is placed in the region before the transcription termination site or the 3′ ITR in the antisense strand of the GOI. The dsRNAs expressed from the DI particles are destabilized or destroyed by the ribozyme (right panel), while the GOI mRNA is not affected (left panel).

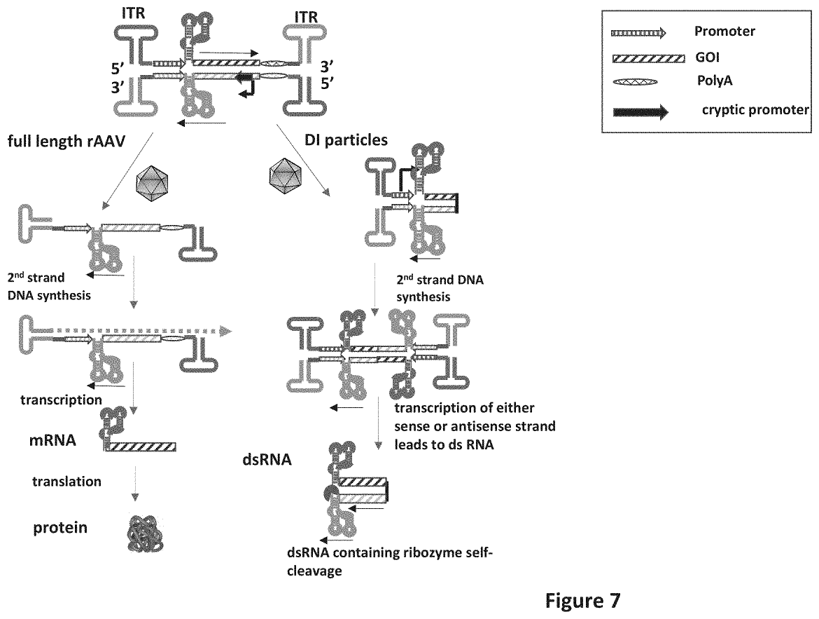

FIG. 8 illustrates the use of an intron with an embedded RDDD for controlling interference by antisense RNAs. The intron is placed in the GOI strand. The RDDD is placed within the antisense strand of the intron. Using an intron with an RDDD allows more flexibility for placement of the RDDD. An intron can be placed within any gene provided that suitable splice donor/acceptor sites are present or introduced therein. In panel A, the intron is embedded right after transcription initiation site and at the 5′ terminal of the GOI starting codon. In panel B, the intron is embedded in the coding region of the GOI.

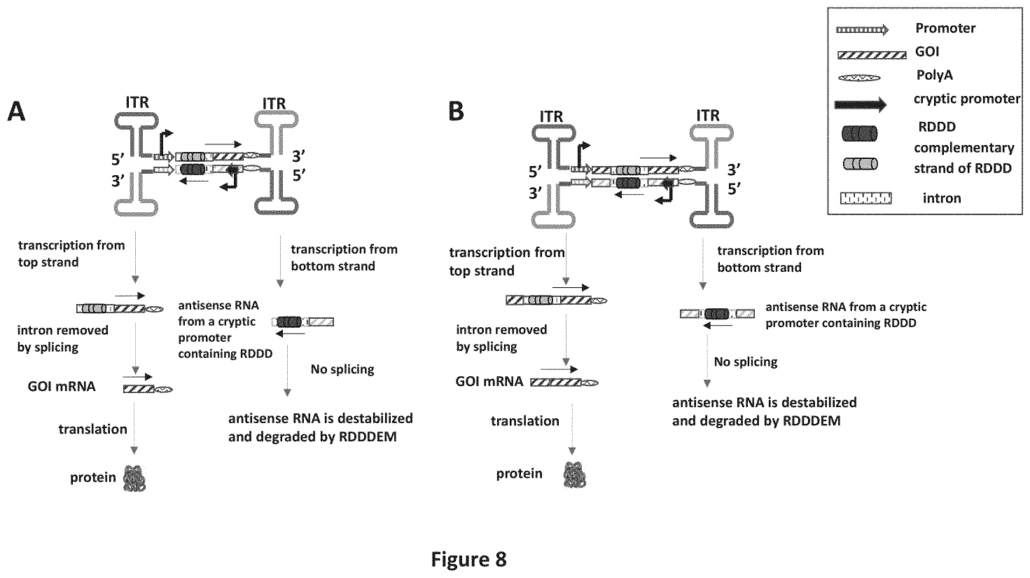

FIG. 9 illustrates the use of an intron with an embedded RDDD for controlling interference by dsRNAs. The intron is in the GOI strand. The RDDD is in the antisense strand of the intron. Using an intron with an RDDD allows more flexibility in placement of the RDDD. An intron can be placed in the gene at any site match exon boundary consensus sequences. In panel A, the intron is embedded right after transcription initiation site and at the 5′ terminal of the GOI starting codon. In panel B, the intron is embedded in the coding region of the GOI.

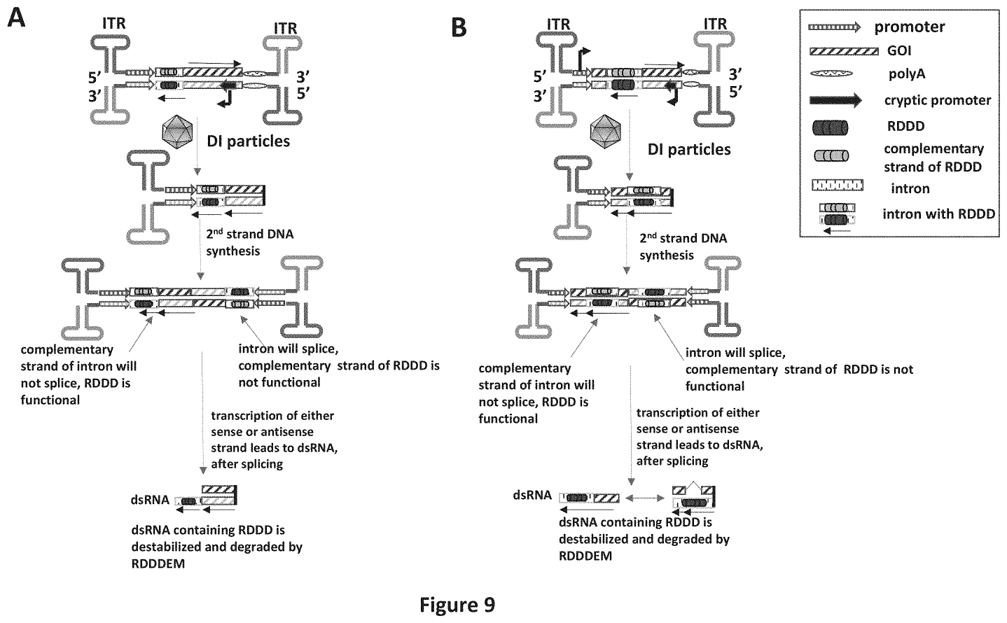

FIG. 10 illustrates three different pathways for managing/controlling antisense RNAs and dsRNAs by expression of RDDDEMs in host cells. The use of endogenously expressed RDDD such as miRNA and siRNA allow the elimination of dsRNA and antisense RNA from vectors.

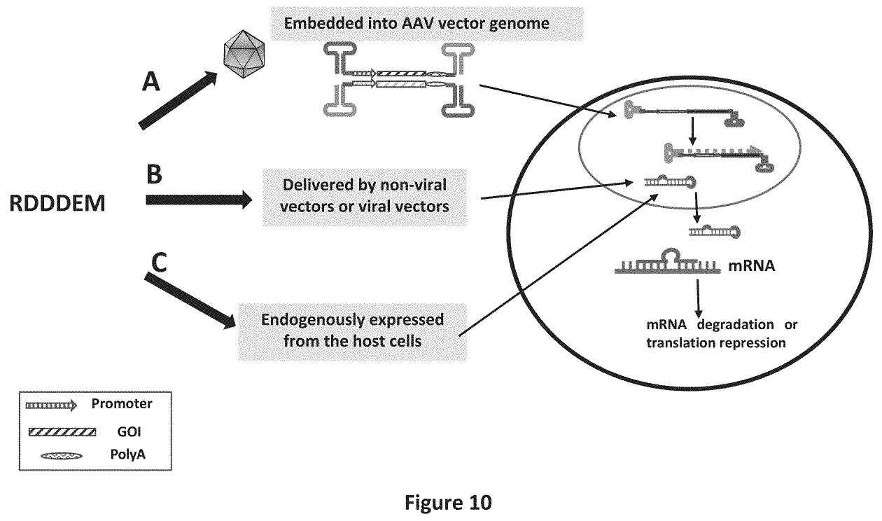

FIG. 11 illustrates the use of an intron with an embedded polyA sequence. Multiple poly A sequences can be used. The intron is placed in close proximity to the cryptic promoter to stop transcription of the antisense RNA against the GOI (right panel), while GOI expression is not affected since intron(s) in GOI is removed during RNA splicing.

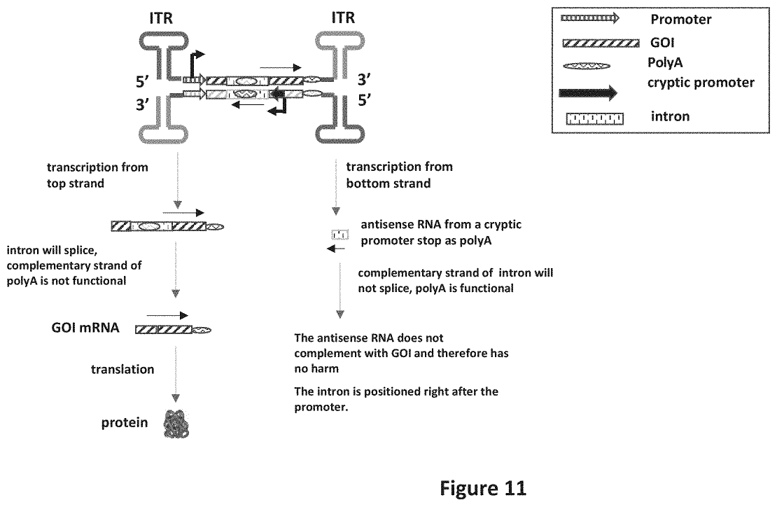

FIG. 12 depicts an exemplary improved parvoviral vector by incorporating an RDDD to reduce or eliminate the effects of dsRNA. The RDDD site is placed in the location before the transcription termination site or the 3′ ITR of the GOI antisense strand. The dsRNAs expressed from the defective interference (DI) particles are destabilized or destroyed by RDDDEM (right panel) while the GOI mRNA is not affected (left panel). The location of RDDD can vary as long as it is only transcribed from the DI particles but not from the full-length vectors. The second feature is the transcription stop sequence (poly A site) added to the opposite strand of the promoter. In the sense strand, the polyA site is in the opposite orientation of the promoter and therefore is not active. In the DI particles formed from AAV preparation, the readthrough from the promoter is stopped. It prevents a situation that a promoter without linked transcription sequences may activate a cellular gene when AAV integrates. The transcription stop site (i.e. poly A) does not affect normal AAV transcription. It helps improve AAV vector safety.

### DETAILED DESCRIPTION

### Definitions

As used herein, the term “parvovirus” refers to any member of the subfamily Parvovirinae, including autonomously-replicating parvoviruses and members of the Dependoparvovirus genus. Autonomously-replicating parvoviruses include members of the genera Amdoparvovirus, Aveparvovirus, Bocaparvovirus, Chapparvovirus, Copiparvovirus, Erythroparvovirus, Protoparvovirus, Tetraparvovirus. Exemplary autonomous parvoviruses include, but are not limited to, minute virus of mice (MVM), bovine parvovirus (BPV), canine parvovirus (CPV), chicken parvovirus, feline panleukopenia virus, feline parvovirus (FPV), goose parvovirus (GPV), porcine parvovirus (PPV), Bocavirus, B19 virus, rat virus (RV), H-1 virus (H-1). Other autonomous parvoviruses are known to those skilled in the art. See, e.g., King A. M. Q., Adams M. J., Carstens E. B. and Lefkowitz E. J. (2012) Virus taxonomy: classification and nomenclature of viruses: Ninth Report of the International Committee on Taxonomy of Viruses. San Diego: Elsevier.).

The genus Dependoparvovirus includes the adeno-associated viruses (AAVs), including but not limited to, AAV type 1 (AAV-1), AAV type 2 (AAV-2), AAV type 3 (AAV-3), AAV type 4 (AAV-4), AAV type 5 (AAV-5), AAV type 6 (AAV-6), avian AAV, bovine AAV, canine AAV, equine AAV, ovine AAV, and the like. The parvovirus particles, capsids and genomes of the present application are preferably from AAV.

Parvoviruses for use in the present application further include new variants from genetic engineering having further modifications in the PV capsid gene, nonstructural genes, inverted terminal repeats (ITRs), left-end hairpins (LEHs), right-end hairpins (REHs). The parvovirus vectors of the present invention are useful for the delivery of nucleic acids to cells both in vitro and in vivo. In particular, the inventive vectors of the present application may be advantageously employed to deliver or transfer nucleic acids to animal cells. Nucleic acids of interest (NAOIs) include nucleic acids encoding RNAs, peptides and proteins, preferably therapeutic (e.g., for medical or veterinary uses) or immunogenic (e.g., for vaccines) peptides or proteins. It may also provide DNA template sequences for gene editing and/or aptamers for targeted delivery.

The term “hybrid parvovirus,” as used herein, refers to a parvovirus genome encapsidated within a different (i.e., another, foreign, exogenous) parvovirus capsid. Alternatively stated, the hybrid parvovirus has a parvovirus genome encapsidated within a different parvovirus capsid. As used herein, by “different” it is intended that the parvovirus genome is packaged within another parvovirus capsid, e.g., the parvovirus capsid is from another parvovirus serotype or from an autonomous parvovirus.

The term “parvovirus ITR,” as used herein, refers to inverted terminal repeats from any parvovirus, which functions in supporting parvovirus replication, encapsidation, rescue, integration etc. Parvovirus inverted terminal repeats (ITRs) are also referred as left-end hairpins (LEHs), right-end hairpins (REHs) when their 5′ ITR and 3′ ITR are different. An “AAV ITR” refers to an inverted terminal repeat flanking the AAV genome. Parvovirus ITRs and AAV ITRs can include ITRs from any parvovirus or any parvovirus serotype, and can further include ITRs with mutations that support AAV replication, encapsidation, rescue and/or integration similar to a wild type ITR.

The term “AAV serotype,” as used herein, refers to any capsids packaged with a genome with at least one AAV ITR. It includes any AAV serotypes found in nature or any engineered or chemically modified capsids that can package AAV genomes. It includes biologically or chemically modified capsids.

The terms “short hairpin DNA” and “shDNA,” are used interchangeably herein with reference to a shDNA as described in US 2018/0298380.

The term “scAAV,” as used herein, refers to a single stranded AAV vector containing a double-stranded region generated by the absence of a terminal resolution site (TR) from one of the ITRs of the AAV, wherein absence of the TR prevents the initiation of replication at the vector terminus where the TR is not present. An scAAV vector typically contains a wild-type (wt) AAV TR at each end and a mutated TR (mTR) in the middle, connectively joined to the AAV TRs by the double stranded region. The terms “mTR” and “mITR,” are used interchangeably herein to mean a mutant inverted terminal repeat as described in U.S. Pat. No. 7,465,583.

The phrases “covalently closed end domain,” “cce domain,” single stranded covalently closed end domain,” and SS-CCE domain” are used interchangeably with reference to a closed single stranded region that is formed at the end of a double-stranded (DS) domain and connects the sense strand of the DS domain to the antisense strand of the DS domain.

The phrase “covalently closed end (cce) parvovirus,” as used herein, refers to a linear parvovirus genome that is packaged into a parvovirus capsid, the parvovirus genome comprising self-complementary DNA sequences forming a pair of hairpin structures at the 5′ and 3′ ends, a double-stranded domain (herein referred to as the “DS domain”) between the 5′ and 3′ ends, and a SS-CCE end. The DS domain is comprised of self-complementary sequences annealing to each other in the genomic DNA. The SS-CCE end comprises non-complementary sequences comprising a closed single stranded region connecting the annealed portions in the DS DS domain. The capsid can be from any parvovirus, including any parvovirus serotype. In preferred embodiments, the cce parvovirus (ccePV) is a cce adeno-associated virus (cceAAV). Self-complimentary (sc) parvovirus vector and scAAV are similar to ccePV and cceAAV in vector genome configuration but they are made by different methods.

The DNA strand in the DS domain may be perfectly complementary or partially complementary over the length of DS domain, such that the complementary sequences can anneal to one another to form stable duplex regions and may form bulged or looped structure in regions of non-complementarity. The regions of non-complementarity may include deletions or insertions in one or both DNA strands such that unique single stranded DNA region(s) may be formed following annealing of the DNA strand to itself. The resulting stem structure(s) may comprise at least 5% of the length of the DS domain. The difference between a cceAAV and an scAAV is that a cceAAV can be more broadly defined in a manner that does not require a mutant TR (mTR). The scAAV is representative of a species within a larger cceAAV genus described herein, which has a unique cce end in the form of a mutant ITR (mITR) or shDNA sequence. The method for preparing scAAV cannot produce the cceAAV vector defined here. However, the method of the present application has the further advantage of providing a more efficient means for producing scAAVs, since the new intermediate template molecules employ two fully functional ITRs, which can be more efficiently replicated in the producer cells as described above.

The term “aptamer” refers to an oligonucleotide or peptide molecule that binds to a specific target molecule. Aptamers are typically created by a selection process utilizing a large random sequence pool and have a variety of research, industrial and clinical applications. For example, aptamers can be combined with ribozymes to self-cleave in the presence of their target molecule. Moreover, natural aptamers are known to exist in riboswitches. Besides the traditional function of selectively binding a target ligand, an “aptamer” as used herein may more broadly include deoxyribozymes, including DNA enzymes, DNAzymes, and catalytic DNAs comprising DNA oligonucleotides capable of performing specific enzymatic reactions.

The term “miRNA,” as used herein, refers to a microRNA (abbreviated miRNA) is a small non-coding RNA molecule (containing about 22 nucleotides) found in plants, animals and some viruses, that functions in RNA silencing and post-transcriptional regulation of gene expression. miRNAs function via base-pairing with complementary sequences within mRNA molecules. As a result, these mRNA molecules are silenced, by one or more of the following processes: (1) Cleavage of the mRNA strand into two pieces, (2) Destabilization of the mRNA through shortening of its poly(A) tail, and (3) Less efficient translation of the mRNA into proteins by ribosomes.

miRNAs resemble the small interfering RNAs (siRNAs) of the RNA interference (RNAi) pathway, except miRNAs derive from regions of RNA transcripts that fold back on themselves to form short hairpins, whereas siRNAs derive from longer regions of double-stranded RNA. The human genome may encode over 1900 miRNAs. The miRNA refers include many variants that with long or short bases as long as it has similar functions as typical miRNA. The target sequence of an miRNA is referred as miRNA target sequence, miR-TS.

The term “siRNA,” as used herein, refers to small interfering RNA (siRNA), sometimes known as short interfering RNA or silencing RNA, is a class of double-stranded RNA non-coding RNA molecules, 20-25 base pairs in length, similar to miRNA, and operating within the RNA interference (RNAi) pathway. It interferes with the expression of specific genes with complementary nucleotide sequences by degrading mRNA after transcription, preventing translation.

The term “ribozyme” as used herein, refers to RNA molecules that are capable of catalyzing specific biochemical reactions, including RNA splicing in gene expression. The most common activities of natural or in vitro-evolved ribozymes are the cleavage or ligation of RNA and DNA and peptide bond formation. Examples of ribozymes include the hammerhead ribozyme, the VS ribozyme, Leadzyme and the hairpin ribozyme. The ribozymes include any RNA molecules that can cleavage RNA molecules. It includes those constructed artificially.

The terms “RNA destruction/destabilization domain” and “RDDD” are used herein with reference to nucleotide sequences that (1) are located in a RNA molecule, and (2) serve as a destruction/destabilization signal causing the RNA to lose its biological function or be degraded partially or entirely. An RDDD may be an miRNA target sequence, a shRNA target sequence, a siRNA target sequences or a ribozyme target sequence or a combination of them. miRNA target sequences, shRNA target sequences, siRNA target sequences or ribozyme target sequences are the target sequences of the corresponding miRNA, shRNA or siRNA or ribozymes, which can act on the RNA molecules containing their target sequences and degrade the RNA molecules partially or entirely. An RDDD in DNA form encodes its RNA form.

The terms “RNA destruction/destabilization domain execution molecule” “RDDDEM,” as used herein, refers to molecules that can affect the RNA molecules containing their corresponding RDDD and degrade the RNA molecules partially or entirely. As RDDDEM may be specific or non-specific to an RDDD. An RDDDEM may be an miRNA, shRNA or siRNA or ribozyme or a combination of them. The corresponding miRNA, shRNA or siRNA or ribozyme can be endogenously expressed, delivered in the same vector containing RDDD, delivered by a separate viral vector or non-viral vectors to the target cells.

The present application generally relates to compositions and methods for alleviating the undesirable effects of negative strand DNA encoded antisense RNAs or dsRNAs arising from defective interference particles that can interfere with the expression and/or translation of GOI mRNAs.

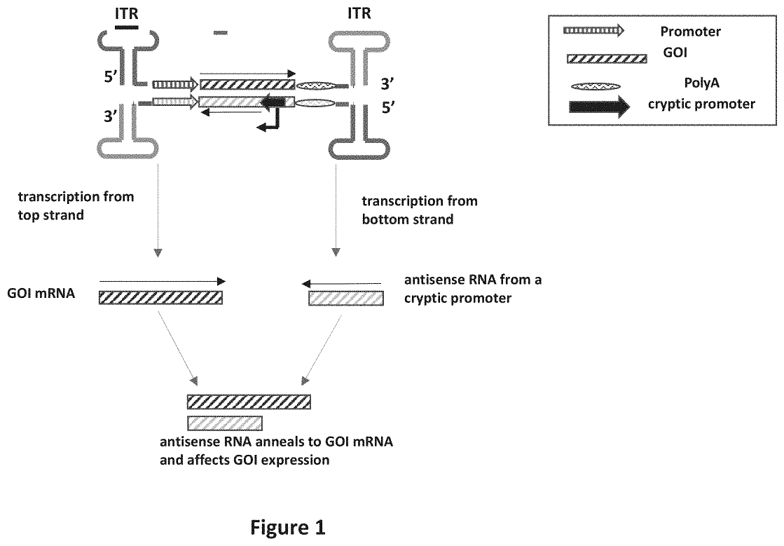

FIG. 1 shows a conventional parvovirus vector, specifically, an adeno-associated virus (AAV) vector comprising ITRs flanking an expression cassette comprising a promoter operably linked to a gene of interest (GOI) and a 3′ polyA signal. Transcription originating from the promoter leads to production of GOI mRNAs. However, it is possible that a cryptic promoter can exist in the antisense strand of the GOI transcription unit, which may facilitate transcription of antisense mRNAs against the GOI mRNA. These antisense RNAs, complementary to the GOI mRNAs, and not necessarily the coding regions only, can negatively affect GOI expression and its intended therapeutic effects when expressed using parvovirus vectors.

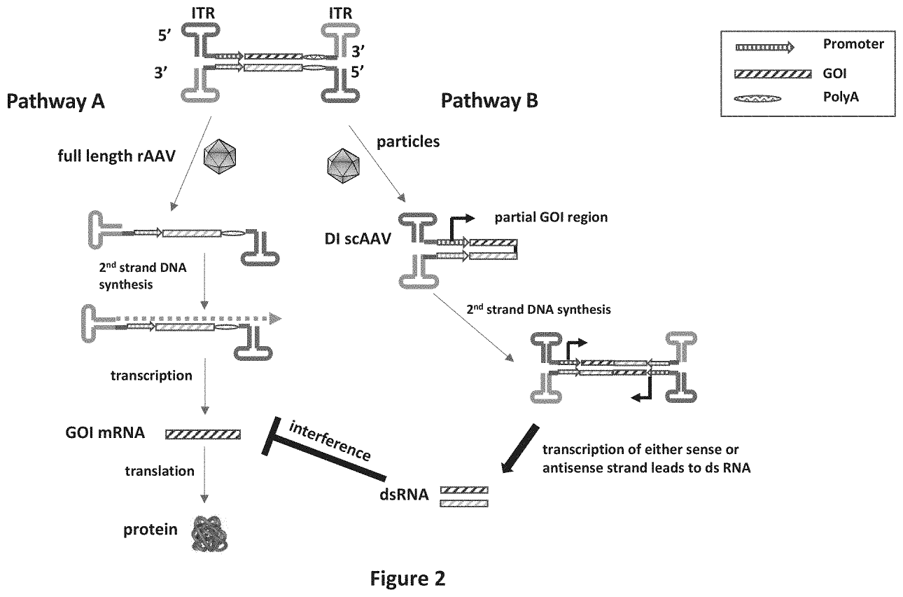

It is known that parvovirus vectors are often contaminated with defective interfering (DI) particles, which may have a self-complimentary genome with partial AAV vector sequences missing. As shown in FIG. 2, DI particles with the promoter region and an incomplete GOI region will allow for the transcription of GOI RNAs forming dsRNAs after the DI particles complete the second stranded DNA synthesis. The dsRNAs can negatively affect wild type GOI expression and reduce the therapeutic effects of parvovirus vectors.

To reduce or eliminate the undesirable effects of antisense RNAs or dsRNAs, the present application introduces an RDDD into the vector design. Thus, in situations where these undesirable antisense RNAs or dsRNAs would be otherwise produced, the RDDD can cause the antisense RNAs and dsRNAs to be destabilized/degraded so they will not negatively affect GOI expression.

In some embodiments, the RDDD comprises one or more target sequences for miRNA, shRNA, siRNA and ribozyme. These target sequences may not be identical to canonical target sequences for a defined miRNA, shRNA, siRNA or ribozyme as long as it serves as the target for the underlying miRNA, shRNA, siRNA or ribozyme. In some embodiments, the target sequences for miRNA, shRNA, siRNA or ribozyme is 50%, 60%, 70%, 80%, 90% or 95% identical to their canonical target sequences. The nucleotide sequences for RDDD may be placed at any region of antisense RNA in the antisense strand of the promoter and GOI. Preferably, when the promoter for GOI is in close proximity of an ITR, RDDD is placed in the same half of genome where the promoter for GOI is located since expression of dsRNA is driving by the promoter of GOI and DI particles expressing dsRNA usually contains less than half of the vector genomes. Potential antisense RNA can be analyzed or determined by RNAseq to define the potential transcription start site and termination site. The RDDD should be placed between such sites. One or multiple RDDDs can be used. In the absence of transcription terminal signal in the antisense strand of the GOI, parvovirus ITR has been shown to function as transcription termination sites.

In one embodiment, one or more RDDDs are placed at the 3′ end of the antisense transcript. In another embodiment, one or more RDDDs are placed in a mid region of the antisense transcript, proximal to the 3′ end. In another embodiment, one or more RDDDs are placed at the 5′ end of the antisense transcript. In yet another embodiment, one or more RDDDs are placed in an intron of the sense strand of the GOI in the reverse orientation. In this case, the one or more RDDDs can be placed in the coding region of the GOI without affecting the final GOI expression. The intron can be in the 5′ untranslated region of the GOI, the 3′ untranslated region of the GOI, or the coding region of the GOI. In another embodiment, one or more copies of a poly A site are placed after the one or more RDDDs. In this case, the RNA sequences upstream of the poly A site(s) are degraded via the one or more RDDDs. To reduce the effects of dsRNAs, the one or more RDDDs are generally placed at the 3′ end of a potential dsRNA. Since dsRNAs shares the same promoter used expressing the GOI, in one embodiment, the one or more RDDDs are placed in the sense strand of the GOI using an intron with an RDDD in the reverse orientation. In another embodiment, the one or more RDDDs are placed in the antisense strand of the promoter, just before the transcription termination site on the antisense strand of the GOI.

It is well-established that nearly all splice sites of introns conform to consensus sequences (matrices). These consensus sequences include nearly invariant dinucleotides at each end of the intron, GT at the 5′ end of the intron, and AG at the 3′ end of the intron.

Splice site consensus sequences for U2 (major class) introns in pre-mRNA generally conform to the following consensus sequences: 3′ splice sites: CAG|G, 5′ splice sites: MAG|GTRAGT where M is A or C and R is A or G. Generally, MAGG nucleotides can be explored for intron insertion between MAG and G. Additional sites can be used to insert a intron containing RDDD or poly A sequences provided that it is functional for RNA splicing.

In one embodiment, an intron of the vector of the present application contains one or multiple polyA sites in the antisense strand. In this configuration, there is no transcription of antisense RNA after the polyA signal. Therefore, this eliminates the production of antisense transcripts against the GOI before the intron (using “GOT” as the reference strand). In yet another embodiment, one or more RDDDs are placed before the polyA sites of the intron in the antisense orientation of the intron. This allows for antisense RNAs against the GOI after the intron (using “GOT” as the reference strand) to be degraded by RDDDEMs against the GOI.

Alternatively, poly A site or transcription termination sites can be placed in the antisense strand of the promoter. In one embodiment, one or multiple polyA sites are located between the promoter and transcription initial size. In the sense stranded, polyA is not functional, it is functional in the antisense stranded. Therefore, this gives the DI particles with a promoter will have a functional polyA site after the conversion of DI particles to a dimer. In yet another embodiment, one or multiple polyA sites are located between the promoter and the enhancers, it is in the antisense orientation as described above. In yet another embodiment, one or multiple polyA sites are located before the promoter, it is in the antisense orientation with regard to the promoter as described above. The main utility of such arrangement of poly A sequence or sites is to prevent promoter read-through in the DI particles.

An RDDD is the biological target for a corresponding RDDDEM. To ensure that antisense RNAs or dsRNAs made from a recombinant parvovirus vector are destabilized or degraded, RDDDEMs must be active in cells transduced with the RDDD-containing parvovirus vectors of the present application. There are several ways that RDDDEMs can be made available for such targeting. First, regulatory RNAs, such as miRNAs, shRNAs, siRNAs or ribozymes may be endogenously available for targeting RDDDs. Second, miRNAs, shRNAs, siRNAs or ribozymes may be expressed from corresponding parvovirus vectors in the sense strand or antisense strand using upstream promoters. Alternatively, miRNAs, shRNAs, siRNAs or ribozymes may be delivered by separate viral vectors, including secondary parvovirus vectors, adenovirus vectors, herpes vectors etc. or they may be delivered to the target cells by non-viral vectors or transfection agents, such as liposomes.

In some embodiments, RDDD and RDDDEM may be used for tissue specific expression or as inducible expression system, parvovirus/AAV vectors are intentionally designed to express antisense or dsRNA to suppress the GOI expression. Such antisense and dsRNA are under the control of RDDD. Subsequently, RDDDEM is induced so the effects of antisense RNA and dsRNA is neutralized, while the expression of the GOI is restored. In one embodiment, the expression of RDDDEM is inducible, resulting an inducible expression vector. In yet another embodiment, the expression of RDDDEM is tissue specific, resulting a tissue specific expression vector.

In some embodiments, a combination of RDDDEM vectors and GOI vectors with RDDD elements is used to transduce target cells together under conditions where the RDDDEM is constitutively expressed. In this case, GOI is expressed normally because RDDDEM expression is not suppressed and RDDDEM will degrade antisense RNAs and dsRNAs. Conversely, when RDDDEM expression is suppressed by inducible agents (in an inducible system) or when RDDDEM expression is restricted in a tissue specific manner (using tissue specific promoters), GOI expression will be inducibly silenced or silenced in a tissue specific manner. Therefore, expression of the GOI can be controlled and regulated.

miRNAs as RDDD Sources

MicroRNAs (miRNAs) are small endogenous non-coding RNA sequences of approximately 21 bp that post-transcriptionally regulate gene expression of about more than 60% of human protein-coding genes. They are involved in most of cellular processes, including development, differentiation, proliferation and apoptosis. miRNAs are highly conserved between species and are specifically expressed at particular levels as a function of e.g., tissue, lineage or differentiation state. More than 2500 unique mature human miRNAs have been identified so far. All such miRNAs can be utilized in the current invention. Individual miRNA species can vary widely in copy number ranging from less than 10 to more than 30000 copies per cell. Besides their tissue-specific expression profiles, several miRNAs are dysregulated in cancer, infectious diseases or diseases of the heart and liver, which can be exploited as RDDDEMs for use in the present application.

MicroRNAs are usually processed from a precursor molecule (pri-microRNA) that folds into a hairpin structure with imperfectly base-paired stems. Pri-microRNAs are further processed by nuclear and cytoplasmatic cleavage proteins, resulting in a short RNA duplex. One strand of the duplex, the guide strand (microRNA), is selected based on the relative free energies of the microRNA duplex ends and is loaded into a multi enzyme complex, the RNA-induced silencing complex (RISC). The less common product is defined as the passenger strand (microRNA), which is assumed to be degraded. Alternatively, both strands of the RNA duplex, namely the 5′ strand (miR-5p) and the 3′ strand (miR-3p) become mature functional microRNAs. The mature miRNA is associated with an Argonaute (AGO) family protein that constitutes the core of the RISC and functions by base-paired binding to the corresponding target site located in the mRNA, resulting in repression of protein synthesis. Complete complementarity of microRNA and its target site leads to endonucleolytical central cleavage of the microRNA/mRNA-duplex by AGO2, using a mechanism similar to RNA-interference mediated by siRNAs. In plants, this mechanism is the predominant one, while the prevalent mechanism in animals involves binding with incomplete complementarity, resulting in inhibition of translation and/or initiation of mRNA degradation. In contrast to plants, whose miRNA target sites are mostly located in protein-coding regions, target sites in animals are often found as repeats in the 3′ UTR of mRNAs.

Several important rules relating to the interaction between a miRNA and its target site were determined by experimental and bioinformatic analyses. The mRNA targeting specificity of a miRNA is determined by a perfect match between the seed sequence, a conserved sequence which is usually situated at positions 2-7 or 2-8 of the 5′-end of the miRNA, with a corresponding sequence in the mRNA. An adenine opposite to position 1 of the microRNA and an adenine or uracil opposite to position 9 of the miRNA are not essential, but increase the efficacy of binding. MicroRNAs exhibiting the same seed sequence belong to the same miRNA family and can regulate the same mRNA targets. A single miRNA species can regulate the production of hundreds of proteins, most likely by recognizing the same seed-matched sequence in the mRNA. A second common characteristic of endogenous microRNA-mRNA interactions comprises nucleotide mismatches in positions 9-12 in the microRNA, which most likely prevents an AGO2-mediated cleavage of the target mRNA. A third characteristic is that matches within the seed region alone are not always sufficient to induce gene repression; stabilization of microRNA binding may need additional complementarity in the 3′ part of the microRNA. In particular, if seed matching is suboptimal, nucleotides at miRNA positions 13-16 become important. Thus the 3′ portion can help to compensate for a single nucleotide mismatch in the seed region, as experimentally confirmed for let7 sites in Caenorhabditis elegans lin-4 and for miR-196 sites in mammalian Hoxb8 mRNA. Additional factors can influence binding stability and thereby the efficacy of microRNA-mediated gene regulation. For example, a more effective repression can result from an AU-rich nucleotide composition near the target site. Additionally, more than 15 nucleotides between an miRNA target sequence (miR-TS) and the stop codon may reduce competition between proteins involved in translation and miRNA-mediated silencing, respectively.

Endogenously expressed miRNAs can also be used to specifically modulate the expression of an exogenous GOI in the form of a therapeutic cDNA. In particular, miRNA target sites or miR-TSs can serve as targets for specific miRNA-mediated post-transcriptional silencing of antisense RNAs and dsRNAs. In some embodiments, one or more miR-TSs are inserted into antisense RNAs and dsRNAs capable of being expressed from a parvovirus vector or silenced using the parvovirus vectors of the present application. In this case, the antisense RNAs and dsRNAs are endonucleolytically cleaved, similar to degradation by siRNAs, and the microRNA-RISC is rapidly recycled. Usually the miRNA guide strand mediates targeted RNA repression; thus, a corresponding sequence representing the miR-TS is inserted into the antisense stand of the transgene expression cassette.

If a miRNA is expressed universally in all tissues, antisense RNAs and dsRNAs expressed from parvovirus vectors with the corresponding miR-TS as an RDDD are degraded in all tissues, leading to enhanced GOI expression in all tissues. Many such miRNAs have already been defined. Where miRNAs are expressed in a cell type- and tissue-specific manner, the corresponding miT-TS elements will be functional only in the cells or tissue where the miRNA is expressed. For example, miR-122 is almost exclusively expressed in liver tissue. Thus, in one embodiment, a miR-122-TS is used as an RDDD such that target expression of the GOI in the liver is not affected, since there is no miR-122-TS in the GOI transcript. However, antisense RNAs or dsRNAs containing a miR-122-TS are suppressed and degraded. Therefore, GOI expression in liver tissue using vectors with a miR-122-TS as an RDDD provide improved and enhanced expression relative to unmodified parvovirus vectors. In another embodiment, where a miR-TS for miR-142-3p is used as an RDDD, expression of the GOI in spleen tissues (where the miR-142 is highly expressed) is unaffected, since there is no miR-142-TS in the GOI transcript. Interference of antisense RNAs and ds-RNAs is reduced or eliminated, because they are degraded by the miR-142 transcripts expressed in spleen tissues.

The following list summarizes miRNA target sequences with tissue preferences with applicability in the present application.

In some embodiments, ES-cell specific miR-296 is used as an RDDD.

In some embodiments, miR-21 and miR-22 is used as an RDDD in differentiated ES cells.

In some embodiments, miR-15a, miR-16, miR-19b, miR-92, miR-93, miR-96, miR-130 or miR-130b is used as an RDDD in both ES cells and various adult tissues.

In some embodiments, miR-128, miR-19b, miR-9, miR-125b, miR-131, miR-178, miR-124a, miR-266 or miR-103 is used as an RDDD in mouse brain development.

In some embodiments, miR-9*, miR-125a, miR-125b, miR-128, miR-132 b miR-137, miR-139, miR-7, miR-9, miR124a, miR-124b, miR-135, miR-153, miR-149, miR-183, miR-190, or miR-219 is used as an RDDD in adult brain.

In some embodiments, miR-18, miR-19a, miR-24, miR-32, miR-130, miR-213, miR-20, miR-141, miR-193 or miR-200b is used as an RDDD in lung.

In some embodiments, miR99a, miR-127, miR-142-a, miR-142-s, miR-151, miR-189b or miR-212 is used as an RDDD in spleen.

In some embodiments, miR-133b, miR-133a-3p, miR-1-3p or miR-206 is used as an RDDD in muscle.

In some embodiments, miR-181, miR-223 or miR-142 is used as an RDDD in hematopoietic tissues.

In some embodiments, miR-122a, miR-152, miR-194, miR-199 or miR-215 is used as an RDDD in liver.

In some embodiments, miR-1b, miR-1d, miR-133, miR-206, miR-208b or miR-143 is used as an RDDD in heart.

In some embodiments, miR-30b, miR-30c, miR-18, miR-20, miR-24, miR-32, miR-141, miR-193 or miR-200b is used as an RDDD in kidney.

In some embodiments, miR-7-5p, miR-375, miR-141 or miR-200a is used as an RDDD in pituitary gland.

In some embodiments, miR-205-5p is used as an RDDD in skin.

In some embodiments, miR-16, miR-26a, miR-27a, miR-143a, miR-21, let-7a, miR-7b, miR-30b or miR-30c is used as an RDDD, since these microRNAs are ubiquitously expressed.

The configuration of the miR-TS is an important factor affecting repression efficacy. Based on the described mechanisms of miRNA-induced gene silencing, completely complementary miR-TS sequences will facilitate higher suppression than imperfectly complementary sequences. As perfectly complementary target sequences are endonucleolytically cleaved between microRNA positions 10/11, the miRNA is rapidly recycled. Consequently, complete complementarity reduces the risk of miRNA saturation and induction of undesirable side effects, because the bioavailability of the cognate miRNA to regulate its natural targets is maintained. Careful design of miR-TS sequences is important for achieving optimal results in controlling expression of antisense RNAs and dsRNAs as outlined in FIG. 1 and FIG. 2.

In addition, the number of miR-TS repeats affects miRNA-mediated suppression of antisense RNA or dsRNA expression. In general, an increased number of miR-TSs enhances microRNA-dependent repression of antisense RNA or dsRNA expression. In some embodiments, 2, 3, 4 or 5 identical repeats of miR-TS are used as RDDDs in the parvovirus of the present application. In other embodiments, 6, 7, 8, 9, 10, 11 or 12 identical repeats of miR-TS are used as RDDDs in the parvovirus of the present application. In some embodiments, different miR-TSs are inserted in series in order to control activity of antisense RNAs and dsRNAs in various cell types with different miRNA expression profiles. A combination of miR-TSs corresponding to different miRNAs for a given cell type or tissue can enhance miRNA-mediated antisense or dsRNA suppression, especially if the microRNAs are only expressed at moderate levels. Moreover, employing cooperative miRNAs may reduce the risk of saturating the function of a particular microRNA.

Enhanced microRNA-mediated suppression of antisense RNA or dsRNA expression can also be achieved by combination of microRNA regulation with other regulation systems. In some embodiments, small 4 to 6 nucleotide long spacer sequences are used to separate miR-TS repeats. Introduction of spacers may, in general, reduce steric hindrance of enzyme complexes binding to microRNA/miR-TS duplexes and facilitate better repression. In some embodiments, spacer-free multimeric miR-TSs are used as the RDDDs in the parvovirus vector of the present application. Insertion of shorter spacers might reduce the risk of forming secondary structures around the miR-TS that might disturb base-paring between the microRNA and the miR-TS. The copy number of miR-TSs and the spacing between miR-TSs are also relevant to viral vector construction, inasmuch as they can increase the size of the transgene expression cassette inserted into the vector genome. As miR-TS are small in size (about 22 bp), insertion of tandem repeats of miR-TS, including spacer sequences, generally do not constitute a capacity problem for most viral vectors. However, for some viral vectors with low packaging capacity, such as self-complementary adeno-associated virus (AAV) vectors, keeping down the total length and number of miR-TSs can be an important consideration. In this regard, shortening of miR-TSs may be necessary. In one embodiment, a deletion of up to 5 nucleotides from the 5′ end of the miR-122TS was well tolerated and did not influence its role in mediating antisense RNA or dsRNA suppression.

The location of the miR-TS in the mRNA is also important for its efficacy in controlling the activity of antisense RNAs and dsRNAs. Secondary structures in mRNAs resulting from insertion of miR-TSs can also affect suppression where accessibility to the target mRNA is hindered. In some embodiments, one or more miR-TSs are inserted in the 3′ UTR of mRNAs, preferably near the stop codon. In other embodiments, one or more miR-TSs are inserted in the 5′ UTR. In other embodiments, one or more miR-TSs are inserted in the open reading frame of the GOI. In this case, it is relatively straightforward for antisense RNA suppression, since is expressed from the antisense strand of the GOI. For dsRNAs, the one or more miR-TSs can be placed in the antisense strand of the GOI, or more specifically, in a reverse complementary orientation in an intron in the sense strand of the GOI.

In general, optimal miRNA-mediated repression of the vector-derived antisense RNAs and dsRNAs can be achieved where high expression of the miRNAs occurs, where insertion of multiple copies (e.g., 3-4 copies) of miR-TS with complete complementarity are employed, where the miR-TS insertions are present in sites with low secondary structure, and where the miRNAs and their target sites exhibit high specificity.

In some embodiments, the RDDD comprises miR-206TS. miR206 is highly expressed in the skeletal muscle and is absent in the heart. By contrast, miR-1 shows high sequence homology to miR-206 but is highly expressed in the heart. Thus, an miR-206TS may be mutated by introducing single nucleotide substitutions into the seed region of the microRNA/miR-206TS duplex. The mutated miR-206TS is resistant to miR-1 regulation, but remains fully sensitive to miR-206. This is a result of compensatory effects induced by perfect complementarity of the 3′ portion of miR206 to mutated miR206TS. In vivo antisense RNA and dsRNA expression of AAV9 vectors bearing mutated miR206TSs and miR122TSs may be strongly suppressed in both skeletal muscle and the liver, whereas antisense RNAs and dsRNAs in heart remain unaffected.

In some embodiments, an miR-122TS-containing vector for expressing antisense RNAs or dsRNAs can be combined with a GOI vector without an miR-122TS so that GOI expression will be restricted to liver, where interference by the miR-122 can be eliminated. This approach is different from the single vector described above for achieving miR-TS-mediated tissue specific expression, since the miR-TS(s) regulating activity of the antisense RNAs and dsRNAs is located on a separate construct than the GOI vector without the miT-TS.

In some embodiments, the RDDD comprises a miR-TS for miR31, miR127 or miR143. miR31, miR127 and miR143 are more highly expressed in normal neural cells than in glioma cells. In some embodiments, the RDDD comprises an miR-124TS. miR-124TS is sufficient to repress transgene expression in neuronal cells in vitro and in vivo, whereas expression is unaffected in astrocytes.

In some embodiments, the RDDD comprises an miR-204TS. Parvoviral vectors comprising one or more miR-204TSs can be selectively suppressed in the retinal pigment epithelium (RPE) RPE, where miR204 expression is known to occur. In some embodiments, the RDDD comprises miR-TS for miR-124, a microRNA that is expressed in photoreceptor PRs, but is absent in RPE. In some embodiments, the RDDD comprises a miR-181cTS. miR-181c is a microRNA expressed in retinal ganglion cells and the inner retina.

Insertion of approximately 100 bp sequence of miR-TS can be accomplished by conventional cloning techniques. In general, the small size of miR-TSs avoids packaging constraints that can otherwise limit the scope of transgene constructs for use in parvoviral vectors, so as to provide wide applicability for both viral vectors and viruses.

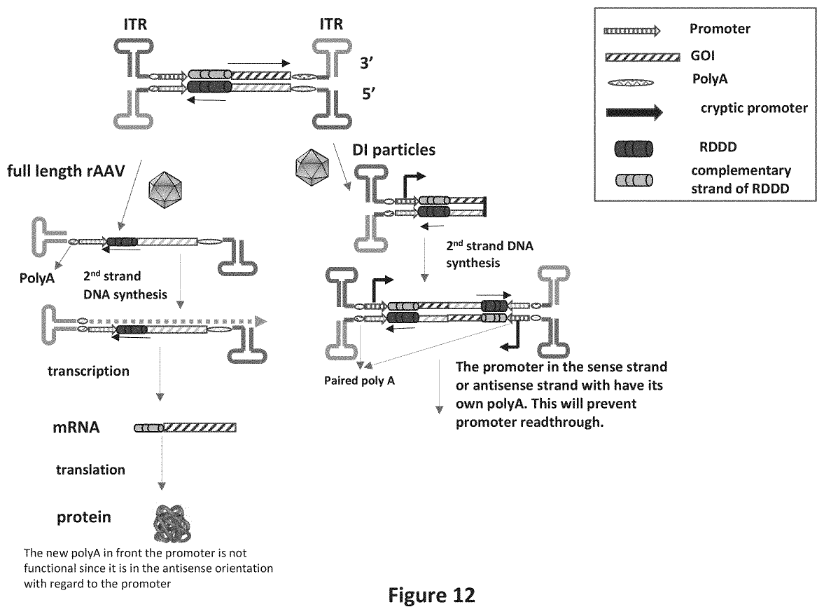

In another embodiment as shown in FIG. 12. An RDDD is included to reduce or eliminate the effects of dsRNA. The RDDD site is placed in the location before the transcription termination site or the 3′ ITR of the antisense strand of the GOI. The dsRNA that is expressed from the defective interference particles are destabilized or destroyed by RDDDEM (right panel) while the GOI mRNA is not affected (left panel). The location of RDDD can vary as long as it is only transcribed from the DI particles but not from the full-length vectors. The second feature is the transcription stop sequence (poly A site) added to the opposite strand of the promoter. In the sense strand, the polyA site is in the opposite orientation of the promoter and therefore is not active. In the DI particles formed from AAV preparation, the read-through from the promoter is stopped. It prevents a situation that a promoter may activate a cellular gene when AAV integrates. The transcription stop site (i.e. poly A) does not affect normal aav transcription. It help improve AAV vector safety.

Ribozymes as RDDD Sources

Self-cleaving ribozymes are a broad category for RNA molecules including twister, twister-sister, hatchet, pistol, Hammerhead ribozyme (HHR) and etc. The HHR structure comprises a “Y”-shape defined by three stems, with stems 1 and 2 interacting with a distal tertiary contact that promotes fast-cleaving mechanisms due to stabilization of the conformation of the active site. By optimal positioning of the required residues, a trans-esterification reaction is catalyzed, resulting in a 5′-product carrying a terminal 2′-3′-cyclic phosphate and a 3′-product with a free terminal 5′-hydroxyl group. Three different topologies of HHRs can be distinguished, depending on the manner in which one of the three stems of the motif connects the HHR to the RNA backbone. More than 10,000 HHR motifs have been discovered by bioinformatics analysis in genomic sequences distributed across all kingdoms of life.

The group I intron-based ribozyme targets and cleaves its substrate RNA and trans-splices an exon attached at its 3′ end (e.g., a therapeutic RNA) onto the cleaved target RNA, resulting in expression of the therapeutic RNA and repression of substrate RNA. Because such ribozymes can specifically target hTERT RNA positive cancer cells, as well also hematopoietic stem cell-derived blood cells, the ribozymes can be modified by inserting target sites for the blood cell-specific miR181a downstream of its 3′ exon. When a ribozyme is used as the RDDD, it can also serve the function of the RDDEM to facilitate a self-catalytic reaction that does not typically require an RDDDEM.

In general, genetic control in eukaryotic systems can be achieved by inserting self-cleaving ribozymes into both the 5′-UTR and 3′-UTR of a given mRNA.

Cleavage of the ribozyme leads to degradation of the mRNA due to removal of the stabilizing 5′-cap or poly(A) structures and therefore to a decrease of gene expression. De-adenylated mRNAs are rapidly degraded by the cytoplasmic exosome. According to this mechanism, ligand-dependent ribozymes have been successfully developed for use in mammalian cells.

Aptazymes are smaller, ligand-inducible self-cleaving RNA-based ribozymes that do not require transcription factors, and can be customized for responsiveness to various small-molecule ligands. The ligand-dependent ribozymes for the triggering of mRNA self-cleavage have been demonstrated in a variety of organisms including mammalian cells, although most have been confined to artificial reporter gene expression regulation. In one embodiment, a theophylline-dependent hammer-head ribozyme (Theo-HHR) is used as an RDDD in the parvovirus vectors of the present application. In some embodiments, a Theo-HHR is inserted into the 3′-end of an antisense RNA or dsRNA to construct a ribozyme-based artificial switch as an RDDD. The joint sequences between an aptamer and an HHR ribozymes can be modulated to affect the function and performance of this riboswitch system.

Chemical regulation of gene expression in mammalian cells can be achieved by embedding a hammerhead ribozyme in the untranslated regions (UTRs) of an mRNA. As such, an HHR may be used to inducibly control activity of antisense RNAs and dsRNAs. In some embodiments, allosterically regulated ribozymes (aptazymes) embedded in the 3′ UTR of antisense RNAs and dsRNAs can be used as riboswitches to chemically regulate gene expression in mammalian cells.

Self-cleaving ribozymes are small RNA motifs found in all kingdoms of life. There are different classes of small self-cleaving ribozymes, including the hammerhead (HHR), hepatitis delta virus (HDV), hairpin, and twister ribozymes. Implementation of engineered HHRs, hepatitis delta virus (HDV), hairpin and twister ribozymes at different positions in the antisense strand [correct?] can promote the cleavage and decay antisense RNAs and dsRNAs so as to reduce their suppression of GOI expression. The integration of aptamers into key structural elements of ribozymes allows for the ligand-responsive control of their self-cleavage activity.

In one embodiment, one or more aptamers (e.g., theophylline aptamer, tetracycline aptamer, guanine aptamer, and MS2 stem-loop/MS2 coat protein aptamer) are connectively linked to one of the interacting stem loops of an HHR (e.g., derived from the satellite RNA of the tobacco ringspot virus (sTRSV) through a short communication sequence. This design concept is based on a ligand-induced change of the ribozyme's secondary structure causing the ribozyme to switch between active and inactive conformational states. Depending on the connection of the communication sequence to the ribozyme, ON- or OFF-type gene switches can be engineered. The sequence identity of the communication sequence strongly determines the performance of an aptazyme, including the basal expression and dynamic range of the gene switch. Therefore, the activity of antisense RNAs and dsRNAs under the control of an aptazyme can be regulated positively- and negatively, respectively.

In some embodiments, the aptazyme is an HHR which is used as an RDDD. In other embodiments, the aptazyme comprises an HDV ribozyme connected to a guanine aptamer, thus resulting in a guanine-responsive gene switch for antisense RNAs and dsRNAs in mammalian cells. In other embodiments, libraries of HDV aptazymes are generated by fusing an aptamer to different sites in a ribozyme. In one embodiment, a guanine aptamer is fused to two different sites of the HDV-like ribozyme drz-Agam-2-1. In another embodiment, the twister ribozyme, which is a ligand-responsive ribozyme with superior self-cleavage activity, is used as an RDDD for suppressing antisense RNAs and dsRNAs. In yet another embodiment, an HHR and a twister ribozyme are used along with miR-TS as the RDDD. In some embodiments, multiple copies of aptazymes are integrated into the 3′UTRs of antisense RNA and dsRNA or 3′UTR in the antisense strand of the parvoviral vector. In some embodiments, aptazymes are simultaneously integrated into the intron and the 3′-UTR. Adjacent RNA sequences and structures should be carefully analyzed when integrating aptazymes, particularly when multiple copies are integrated into single mRNAs. In some embodiments, aptazymes are flanked by spacer sequences to prevent undesired secondary structures and ensure correct folding of individual aptazymes. In some embodiments, bioinformatics tools are used for in silico predictions of RNA secondary structures, and to facilitate aptazyme engineering and integration procedures for designed RNA sequences. Table 1 shows an exemplary list of miR-TS elements that can be used as RDDDs for controlling the activity of antisense RNAs and dsRNAs in different target cells and tissues.

TABLE 1
Exemplary miR-TS elements as RDDDs for controlling antisense
RNA and dsRNAs in different target cells and tissues.
For example, miR-TS for let-7a can be used as RDDD for
controlling antisense RNA and dsRNAs in non-pluripotent
cells; miR-TS for miR-1 can be used as RDDD for controlling
antisense RNA and dsRNAs in heart tissue, etc.

| miR-TS for | Eliminate antisense RNA and dsRNA in cell/tissue |
|---|---|
| let-7a | Non pluripotent cells |
| miR-1 | Heart |
| miR-122 | Liver |
| miR-124 | Neurons |
| miR-126 | Hematopoietic stem and progenitor cells |
| miR-127 | Astrocytes/brain |
| miR-128 | Neuronal differentiated cells |
| miR-130a | Hematopoietic stem and progenitor cells |
| miR-142-3p | Human ES, neural progenitors |
| miR-143 | Astrocytes/brain |
| miR-150 | Differentiated T and B lymphocytes |
| miR-155 | Granulocytes and monocytes, mature dendritic cells |
| miR-181 | Hematopoietic stem cell-and progenitor-derived blood cells |
| miR-181a | Developing T cells |
| miR-181c | Ganglion cells and inner retina |
| miR-204 | Photoreceptors/retinal pigment epithelium |
| miR-206 | Liver, skeletal muscle |
| miR-208a | Heart |
| miR-221 | Cortical inhibitory neurons |
| miR-223 | Granulocytes and monocytes, mature dendritic cells |
| miR-292-3p | Pluripotent cells |
| miR-302a, d | Human ES, neural progenitors |
| miR-31 | Astrocytes/brain |

Functional Advantages of the RDDD of the Present Application

Compared to conventional AAVs carrying miRNAs, shRNAs, siRNAs and ribozyme targeting sequences, the RDDDs of the present application serve a fundamentally different role. Unlike conventional vectors where miRNAs, shRNAs, siRNAs and ribozyme targeting sequences are placed at the 3′ end of the GOI transcripts, the GOI transcripts of the present application are directly regulated by the corresponding miRNAs, shRNAs, siRNAs and ribozymes. In the present application, RDDDs are integrated into antisense or dsRNA. Its orientation is always in the complement strand of the GOI transcripts. The dsRNA against parvovirus vectors are thus produced from its corresponding DI particles, which affect the GOI transcripts. In other words, the RDDD is integrated into an antisense strand such that its orientation is always inversely complementary to the GOI transcript. Accordingly, the functional activity of antisense RNAs and dsRNAs produced from parvoviral DI particles, specifically their negative effects on GOI expression, can be reduced or eliminated.

A chief advantage of vectors carrying RDDDs is their ability to provide long term and stable gene expression by eliminating the negative effects of antisense RNA and dsRNA. Antisense and dsRNA molecules targeting a GOI can directly regulate GOI transcripts and decrease their expression through GOI RNA degradation. In addition, antisense RNAs and dsRNAs are known to induce immune responses and reduce/destabilize GOI expression.

An important feature of the vectors of the present application is that the vectors can be designed to be regulated by endogenous/exogenous miRNAs, siRNAs, shRNAs and ribozymes for controlling antisense RNA and dsRNA expression so that specific expression profiles for a given vector can be achieved. Accordingly, these vectors can be used for gene therapy approaches to prevent immune responses and/or maintain long term transgene expression.

Another aspect of the present application relates to a method for improving production yield of a recombinant parvoviruses, comprising the steps of: inserting a RNA destabilization/destruction domain (RDDD) into the recombinant parvovirus. The recombinant parvovirus comprises: a parvovirus capsid; and a double-stranded viral genome comprising a sense-strand and an antisense-strand, wherein the sense-strand comprises in the 5′ to 3′ direction: a parvovirus terminal repeat at the 5′ end; a coding sequence of a gene of interest (GOI); and a parvovirus terminal repeat at the 3′ end, wherein the RDDD is inserted into the viral genome.

In some embodiments, the RDDD is located in the antisense-strand of the viral genome. In some embodiments, the RDDD is located in an intron of the GOI in a reverse orientation.

### EXAMPLES

### Example 1: Ubiquitously Expressed miR-16-TS for Antisense RNA and dsRNA Elimination

The hsa-miR-16 sequence was obtained from the miRNA registry (Griffiths-Jones, 2004). An RDDD with the miR-16 target sequence was synthesized and cloned into a factor VIII (FVIII) expression construct pAAV-F8 which has a B domain deleted factor VIII under the control of liver specific promoter TTR. An RDDD sequence with an miR-16 sequence inserted between the promoter and FVIII starting codon includes the sequence,

(SEQ ID NO: 1)
TAGCAGCACGTAAATATTGGCG.

The underlined sequence in the GOI strand is designed to match perfectly to a specific miRNA. In the bottom strand, the sequence is perfectly complementary to the specific miRNA, which is miR-16 in this case. The resulting construct is pAAV-F8-miR-16-PG. Only one copy miRNA is used to avoid the long space between the promoter and GOI. The vectors based pAAV-F8-miR-16-PG were produced by triple plasmid transfection method, which is commonly reported. The DI composition was sequenced by pacBio and confirmed the molecules would express dsRNA in vivo. Upon injection of vectors produced by pAAV-F8 and pAAV-F8-miR-16-PG into C57B6 mice, liver tissues were harvested and RNA was extracted from the samples. Quantitative rtPCR analysis confirmed the presence of dsRNA from pAAV-F8-miR-16-PG was reduced from 80% to undetectable level.

### Example 2: Multiple Copies of miR-16-TS for Antisense RNA and dsRNA Elimination

RDDD with four copies miR-16 target sequences was synthesized and cloned into a factor VIII (FVIII) expression construct pAAV-F8 which has B domain deleted factor VIII under the control of liver specific promoter TTR. Since there is no polyA signal in the antisense strand of promoter of GOI, transcripts for antisense RNA and dsRNA will extend beyond the promoter and stop at ITR, which is shown to have the function of a polyA site. RDDD with 4 copies of miR-16 was inserted between the promoter and 5′ ITR including the following sequence:

(SEQ ID NO: 2)
gtacTAGCAGCACGTAAATATTGGCGgcta
gcTAGCAGCACGTAAATATTGGCGgtcagc
TAGCAGCACGTAAATATTGGCGagctgc
TAGCAGCACGTAAATATTGGCGcgta

The underlined sequences in the GOI strand were designed to match perfectly to a specific miRNA. In the bottom strand, these sequences were perfectly complementary to the specific miRNA, which is miR-16 in this case. The resulting construct was pAAV-F8-miR-16-TP. Another ideal position for inserting the RDDD is nucleotides between the promoter and the enhancers, in which the distance usually does not affect the promoter activities. The vectors based pAAV-F8-miR-16-TP were produced by triple plasmid transfection. The DI composition was sequenced by pacBio and confirmed the molecules would express dsRNA in vivo. Upon injection of vectors produced by pAAV-F8-miR-16-TP into C57B6 mice, liver tissues were harvested and RNA was extracted from the sample tissue. Quantitative rtPCR analysis confirmed the presence of dsRNA from vectors produced from pAAV-F8-miR-16-TP was reduced to undetectable level based on three repeated experiments.

### Example 3: miR-16-TS and miR-122 for Antisense RNA and dsRNA Elimination in Liver

Since there is no polyA signal in the antisense strand of promoter of GOI, transcripts for antisense RNA and dsRNA will extend beyond the promoter and stop at ITR, which is shown to have the function of a poly A sequences. In this study, an RDDD with 2 copies of miR-16 and two copies of miR-122

(SEQ ID NO: 3)
(CAAACACCATTGTCACACTCCA

were inserted between the promoter and enhancers and includes the following sequence:

(SEQ ID NO: 4)
gtacCAAACACCATTGTCACACTCCAgcta
gcTAGCAGCACGTAAATATTGGCGgtcagc
CAAACACCATTGTCACACTCCAagctgcTA
GCAGCACGTAAATATTGGCGcgta.

The underlined sequences in the GOI strand are designed to match perfectly to a specific miRNA. In the bottom strand, these sequences are perfectly complementary to the specific miRNA, which are miR-16 and miR-122 in this case. The resulting construct was pAAV-F8-miR-16-122-EP. The vectors based on pAAV-F8-miR-16-122-EP were produced by triple plasmid transfection. The DI composition was sequenced by pacBio and confirmed the molecules would express dsRNA in vivo. Upon injection of vectors produced by pAAV-F8 and pAAV-F8-miR-16-122-EP into C57B6 mice, liver tissues were harvested and RNA was extracted from the samples. Quantitative rtPCR analysis confirmed the presence of dsRNA in AAV-F8 and dsRNA from pAAV-F8-miR-16-122-EP was reduced 75% to undetectable.

### Example 4: miR-16-TS and miR122 for Antisense RNA and dsRNA Elimination in Liver, Embedded in Coding Sequences of GOI

In this study, the RDDD includes two copies of miR-16 and two copies of miR-122 embedded into an intron as reflected in the following sequence:

(SEQ ID NO: 5)
gtaagtgccgtgtgtggttcccgcgggcc
tggcctctttacgggttatggcCAAACAC
CATTGTCACACTCCAgctagcTAGCAGCA
CGTAAATATTGGCGgtcagcCAAACACCA
TTGTCACACTCCAagctgcTAGCAGCACG
TAAATATTGGCGccttgcgtgccttgaa
ttactgacactgacatccactttttcttt
ttctccacag

The synthetic intron is in small letters. The underlined sequences in the GOI strand are designed to match perfectly to a specific miRNA. In the bottom strand, these sequences are perfectly complementary to the specific miRNA, which are miR-16 and miR-122 in this case. The resulting construct is pAAV-F8-miR-16-122-IN. The intron is inserted in a CAG/G sequence in the factor VIII gene. The vectors based on pAAV-F8-miR-16-122-IN were produced by triple plasmid transfection. The DI composition was sequenced by pacBio and confirmed the molecules expressed dsRNA. Upon injection of vectors produced by pAAV-F8 and pAAV-F8-miR-16-122-IN into C57B6 mice, liver tissues were harvested and RNA was extracted from the samples three weeks later. Quantitative rtPCR analysis confirmed the presence of dsRNA from vectors produced from pAAV-F8-miR-16-122-IN is undetectable. Factor VIII expressed in this construct functioned normally in aPTT assay.

### Example 5: miR-16-TS and miR122 for Antisense RNA and dsRNA Elimination in Liver, Embedded in Coding Sequences with Additional Poly A Sequences

To demonstrate embedded function of polyA sequence, the RDDD includes two copies of miR-16 and two copies of miR-122 embedded into an intron as reflected in the following sequence:

(SEQ ID NO: 6)
gtaagtgccgtgtgtggttcccgcgggcc
tggcctctttacgggttatggcCAAACAC
CATTGTCACACTCCAgctagcTAGCAGCA
CGTAAATATTGGCGgtcagcCAAACACCA
TTGTCACACTCCAagctgcTAGCAGCACG
TAAATATTGGCGccttgcgtgccttgaat
tactgaTTTATTcactgacatccactttt
tcatttctccacag

The synthetic intron is shown as small letters. The polyA sequence is presented in the reverse complementary orientation in the coding strand. The underlined sequences in the GOI strand are designed to match perfectly to a specific miRNA. In the bottom strand, these sequences are perfectly complementary to the specific miRNA, which are miR-16 and miR-122 in this case. The resulting construct is pAAV-F8-miR-16-122-IP. The intron is inserted in a CAG/G sequence in the factor VIII gene. The vectors based on pAAV-F8-miR-16-122-IP were produced by triple plasmid transfection. The DI composition was sequenced by pacBio and confirmed the molecules would express dsRNA. Upon injection of vectors produced by pAAV-F8 and pAAV-F8-miR-16-122-IP into C57B6 mice, liver tissues were harvested and RNA was extracted from the samples. Quantitative rtPCR analysis confirmed the presence of dsRNA in vectors produced from pAAV-F8-miR-16-122-IP is reduced 70% to undetectable level. Factor VIII expressed in this construct functioned normally in aPTT assay.

### Example 6: Dual RDDD for miR16-TS and miR122 for Antisense RNA and dsRNA Elimination. Poly a Site in the Antisense Strand of GOI (Factor VIII) Gene

In this study, a dual RDDD with two copies of miR-16 and two copies of miR-122 embedded into an intron includes the following sequence:

(SEQ ID NO: 7)
gtaagtgccgtgtgtggttcccgcgggcc
tggcctctttacgggttatggcCAAACAC
CATTGTCACACTCCAgctagcTAGCAGCA
CGTAAATATTGGCGgtcagcCAAACACCA
TTGTCACACTCCAagctgcTAGCAGCACG
TAAATATTGGCGccttgcgtgccttgaat
tactgacactgacatccactttttctttt
tctccacag

The synthetic intron is in small letters. The underlined sequences in the GOI strand are designed to match perfectly to a specific miRNA. In the bottom strand, these sequences are perfectly complementary to the specific miRNA, which are miR-16 and miR-122 in this case. The intron is inserted in a CAG/G sequence in the factor VIII gene. The second RDDD has the sequences of

(SEQ ID NO: 8)
gtacTAGCAGCACGTAAATATTGGCGgct
agcTAGCAGCACGTAAATATTGGCG,

which is inserted between the enhancer and promoter. The resulting construct is pAAV-F8-miR-16-122-DR. The vectors based on pAAV-F8-miR-16-122-DR were produced by triple plasmid transfection. The DI composition was sequenced by pacBio and confirmed the molecules would express dsRNA in vivo. Upon injection of vectors produced by pAAV-F8 and pAAV-F8-miR-16-122-DR into C57B6 mice, liver tissues were harvested and RNA was extracted from the samples. Quantitative rtPCR analysis confirmed dsRNA from pAAV-F8-miR-16-122-DR derived is undetectable. Factor VIII expressed in this construct functioned normally in aPTT assay.

### Example 7: Ribozymes for Antisense RNA and dsRNA Elimination in which there is No polyA Site in the Antisense Strand of Promoter

RDDD with sLTSV(−) type 3 HHR was synthesized and cloned into pAAV-F8 between the 5′ITR and the promoter. The ribozyme sLTSV(−) type 3 HHR was shown to reduce gene expression up to 60-fold compared to the inactive form in the mammalian expression system. The sequence of a cassette from sLTSV(−) type 3 HHR is

(SEQ ID NO: 9)
5′-TAATTCTAGGCGACTAGTAAACAAA
CAAAGACGTATGAGACTGACTGAAACGCC
GTCTCACTGATGAGGCCATGGCAGGCCGA
AACGTCAAAAAGAAAAATAAAAA-3′

when read from the complementary strand for the GOI. The underlined sequence in the complementary strand of GOI strand are the ribozyme with flanking sequences not underlined. The resulting construct is pAAV-F8-RZ. The vectors based pAAV-F8-RZ were produced by triple plasmid transfection. The DI composition was sequenced by pacBio and confirmed the molecules would express dsRNA in vivo. Upon injection of vectors produced by pAAV-F8 and pAAV-F8-RZ into C57B6 mice, liver tissues were harvested and RNA was extracted from the samples. Quantitative rtPCR analysis confirmed the presence of dsRNA from pAAV-F8-RZ was reduced 90% to undetectable.

### Example 8: Vector Performances with dsRNA Removed

To confirm that dsRNA is controlled in vectors in the above examples 1-7, the expression kinetics of the dsRNA sensors such as MDA5 was analyzed in the control vector pAAV-F8. The upregulation of MDA5 was observed at days 6 and 8 after the control vector administration to HeLa cells (approximately 2 to 3 fold increases). However, all the above designed vectors did not show signs of MDA5 upregulation. The above vectors were injected in hemophilia A mice at a dose of 1e11/viral particles per mouse. The control vector AAV-f8 had the lowest expression. All other vectors have showed factor VIII expression level by ELISA and aPTT assay, which showed improved expression of factor VIII in a range from 50% to 10-fold.

1. A parvovirus vector comprising:
a parvovirus capsid; and
a double-stranded vector genome comprising a sense-strand and an antisense-strand,
wherein the sense-strand comprises in the 5′ to 3′ direction:
a parvovirus terminal repeat at the 5′ end;
a coding sequence of a gene of interest (GOI); and
a parvovirus terminal repeat at the 3′ end,
wherein the vector genome further comprises a RNA destabilization/destruction domain (RDDD).

2. The parvovirus vector of claim 1, wherein the RDDD is located in the antisense-strand.

3. The parvovirus vector of claim 1, wherein the RDDD is located in an intron of the GOI in a reverse orientation.

4. The parvovirus vector of claim 1, wherein the parvovirus vector is an AAV vector.

5. The parvovirus vector of any one of claims 1-4, wherein the RDDD comprises a microRMA.

6. The parvovirus vector of any one of claims 1 to 5, wherein the RDDD comprises SEQ ID NO:1 and/or SEQ ID NO:3.

7. The parvovirus vector of any one of claims 1 to 6, wherein the RDDD comprises two or more copies of SEQ ID NO:1 and/or two or more copies of SEQ ID NO:3.

8. The parvovirus vector of any one of claims 1 to 7, wherein the RDDD comprises the nucleotide sequence selected from the group consisting of SEQ ID NOS: 2, 4, 5, 6 and 7.

9. The parvovirus vector of any one of claims 1-9, further comprising a second RDDD.

10. The parvovirus vector of claim 9, wherein the second RDDD comprises SEQ ID NO:8.

11. The parvovirus vector of any one of claims 1-4, wherein the RDDD comprises a ribozyme.

12. The parvovirus vector of claim 11, wherein the ribozyme comprises SEQ ID NO:9.

13. A method for improving production yield of a recombinant parvovirus, comprising the steps of:
inserting a RNA destabilization/destruction domain (RDDD) into the recombinant parvovirus,
wherein the recombinant parvovirus comprises:
a parvovirus capsid; and
a double-stranded viral genome comprising a sense-strand and an antisense-strand, wherein the sense-strand comprises in the 5′ to 3′ direction:
a parvovirus terminal repeat at the 5′ end;
a coding sequence of a gene of interest (GOI); and
a parvovirus terminal repeat at the 3′ end,
wherein the RDDD is inserted into the viral genome.

14. The method of claim 13, wherein the RDDD is located in the antisense-strand of the viral genome

15. The method of claim 13, wherein the RDDD is located in an intron of the GOI in a reverse orientation.
